SEQUENCE LISTING (21 sequences)

SEQ ID NO: 1 - DNA, 20 bp, Artificial Sequence
  CpG
tccatgacgttcctgacgtt

SEQ ID NO: 2 - DNA, 14 bp, Artificial Sequence
  LNA sequence
ggaaggaaggaagg

SEQ ID NO: 3 - DNA, 34 bp, Artificial Sequence
  LNA-CpG sequence
tccatgacgttcctgacgttggaaggaaggaagg

SEQ ID NO: 4 - DNA, 20 bp, Artificial Sequence
  Oligonucleotide
tccatgacgttcctgacgtt

SEQ ID NO: 5 - DNA, 18 bp, Artificial Sequence
  Oligonucleotide
tctcccagcgtgcgccat

SEQ ID NO: 6 - DNA, 30 bp, Artificial Sequence
  Oligonucleotide
accgatgacgtcgccggtgacggcaccacg

SEQ ID NO: 7 - DNA, 20 bp, Artificial Sequence
  Oligonucleotide
tccatgacgttcctgatgct

SEQ ID NO: 8 - DNA, 13 bp, Artificial Sequence
  Oligonucleotide
ctctctctctctc

SEQ ID NO: 9 - DNA, 14 bp, Artificial Sequence
  Oligonucleotide
ccttccttccttcc

SEQ ID NO: 10 - DNA, 13 bp, Artificial Sequence
  Oligonucleotide
gagagagagagag

SEQ ID NO: 11 - DNA, 11 bp, Artificial Sequence
  Oligonucleotide
ctctctctctc

SEQ ID NO: 12 - DNA, 18 bp, Artificial Sequence
  Oligonucleotide
ctctctctcctctctctc

SEQ ID NO: 13 - DNA, 11 bp, Artificial Sequence
  Oligonucleotide
ctctctctctc

SEQ ID NO: 14 - DNA, 34 bp, Artificial Sequence
  Oligonucleotide
tccatgacgttcctgacgtttgagagagagagag

SEQ ID NO: 15 - DNA, 34 bp, Artificial Sequence
  Oligonucleotide
tccatgagcttcctgagtcttgagagagagagag

SEQ ID NO: 16 - DNA, 13 bp, Artificial Sequence
  Oligonucleotide
gagagagagagag

SEQ ID NO: 17 - DNA, 20 bp, Artificial Sequence
  Oligonucleotide
tccatgagcttcctgagtct

SEQ ID NO: 18 - DNA, 35 bp, Artificial Sequence
  Oligonucleotide
tccatgacgttcctgacgtttggaaggaaggaagg

SEQ ID NO: 19 - DNA, 35 bp, Artificial Sequence
  Oligonucleotide
tccatgagcttcctgagtcttggaaggaaggaagg

SEQ ID NO: 20 - DNA, 35 bp, Artificial Sequence
  Oligonucleotide
aggatgacgttggagacgtttggaaggaaggaagg

SEQ ID NO: 21 - DNA, 35 bp, Artificial Sequence
  Oligonucleotide
aggatgagcttggagagtcttggaaggaaggaagg

(19) United States
(12) Patent Application Publication
(10) Pub. No.: US 2006/0194311 A1
(43) Pub. Date: Aug. 31, 2006

(54) Lna-cpg conjugates

(76) Inventor: Ian Richard Catchpole, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/538,129

(22) PCT Filed: Dec. 4, 2003
(86) PCT No.: PCT/EP03/13803

§ 371 Date: Feb. 27, 2006

(30) Foreign Application Priority Data
Dec. 6, 2002 (GB) 0228540.1

(51) Int. Cl. C12N 15/63; C07K 14/435

(52) U.S. Cl. 435/320.1; 530/352

(57) ABSTRACT

The present invention relates to novel compositions comprising DNA attached to an immunostimulatory oligonucleotide (CpG) via a locked nucleic acid oligonucleotide of a defined sequence. In particular the present invention provides compositions comprising a plasmid containing a gene encoding a protein of interest, wherein said plasmid may be introduced to a tissue or cell and the gene expressed, complexed to the LNA-CpG. Also provided is a method of co-synthesis of CpG and LNA to form compositions for use as adjuvants with DNA plasmid vaccines.

The present invention relates to novel compositions comprising DNA attached to an immunostimulatory oligonucleotide (CpG) via a locked nucleic acid oligonucleotide of a defined sequence. In particular the present invention provides compositions comprising a plasmid containing a gene encoding a protein of interest, wherein said plasmid may be introduced to a tissue or cell and the gene expressed, complexed to the LNA-CpG. Also provided is a method of co-synthesis of CpG and LNA to form compositions for use as adjuvants with DNA plasmid vaccines.

Plasmid based delivery of genes, particularly for immunisation or gene therapy purposes is known. For example, administration of naked DNA by injection into mouse muscle is outline by Vical in International Patent Application WO90/11092.

Johnston et al WO 91/07487 describe methods of transferring a gene to vertebrate cells, by the use of microprojectiles that have been coated with a polynucleotide encoding a gene of interest, and accelerating the microparticles such that the microparticles can penetrate the target cell.

DNA vaccines usually consist of a bacterial plasmid vector into which is inserted a strong viral promoter, the gene of interest which encodes for an antigenic peptide and a polyadenylation/transcriptional termination sequences. The gene of interest may encode a full protein or simply an antigenic peptide sequence relating to the pathogen, tumour or other agent which is intended to be protected against. The plasmid can be grown in bacteria, such as for example E.coli and then isolated and prepared in an appropriate medium, depending upon the intended route of administration, before being administered to the host. Following administration the plasmid is taken up by cells of the host where the encoded peptide is produced. The plasmid vector will preferably be made without an origin of replication which is functional in eukaryotic cells, in order to prevent plasmid replication in the mammalian host and integration within chromosomal DNA of the animal concerned.

There are a number of advantages of DNA vaccination relative to traditional vaccination techniques. First, it is predicted that because of the proteins which are encoded by the DNA sequence are synthesised in the host, the structure or conformation of the protein will be similar to the native protein associated with the disease state. It is also likely that DNA vaccination will offer protection against different strains of a virus, by generating cytotoxic T lymphocyte response that recognise epitopes from conserved proteins. Furthermore, because the plasmids are taken up by the host cells where antigenic protein can be produced, a long-lasting immune response will be elicited. The technology also offers the possibility of combing diverse immunogens into a single preparation to facilitate simultaneous immunisation in relation to a number of disease states.

Helpful background information in relation to DNA vaccination is provided in Donnelly et al “DNA vaccines” Ann. Rev Immunol. 1997 15: 617-648, the disclosure of which is included herein in its entirety by way of reference.

Despite the numerous advantages associated with DNA vaccination relative to traditional vaccination therapies there is nonetheless a desire to develop improvements which will serve to increase the immune response induced by the protein which is encoded by the plasmid DNA administered to an animal. The present invention addresses these issues.

Locked nucleic acid (LNA) is an analogue of RNA or DNA. The term LNA is used to describe both nucleotide monomers, in which the ribose ring is constrained by a methylene linkage between the 2′-oxygen and the 4′-carbon, and also oligonucleotides that contain one or more monomers of locked nucleic acid. The methylene bridge linkage can be through oxygen, (oxy-LNA), sulphur, (thio-LNA) and amine, (amino-LNA). The confirmation restriction increases binding affinity for complementary sequences (Dwaine A. Braasch and David R. Corey, Chemistry and Biology 8 (2001) 1-7). The introduction of LNA monomers into DNA or RNA oligonucleotides increases affinity for complementary DNA or RNA sequences, ie. measured as thermal stability of duplexes, eg. melting temperature, (Tm), increases in the range of 3-8° C., depending on the actual base, per LNA monomer present in the oligonucleotide., (Dwaine A. Braasch and David R. Corey, Chemistry and Biology 8 (2001) 1-7). Synthesis of LNA is described in International Patent Application No. WO99/14226.

Linking peptide and other material (eg. fluorescent labels such as rhodamime) to DNA plasmid by means of a Peptide nucleic acid oligonucleotide is known (U.S. Pat. No. 6,165,720). These have also been used to transfect cells. Although reports have been made of PNA/DNA/PNA triplexes surviving quite harsh conditions (50), such studies were only performed on short DNA oligonucleotides and not upon large supercoiled plasmid DNA, where stability of bound PNA is not sufficent to enable a PNA-coupled fluorophore or peptide to remain attached to plasmid DNA when administered in a pharmaceutical or vaccine formulation, especially for (particle mediated immunotherapeutic delivery) PMID.

WO 02/102825 discloses the use of LNA linked functional moeities as adjuvants for plasmid DNA vaccines.

The present invention provides a novel LNA-immunostimulatory oligonucleotide compositions that may be used enhance the immune response stimulated by DNA vaccines. The immunostimulatory oligonucleotides in the present invention are oligonuclotides that comprise at least one unmethylated CG dinucleotide (CpG).

In the first aspect of the present invention there is provided a novel composition comprises a oligonucleotide comprising an unmethylated CG dinucleotide (CpG) of the following sequence:

5′-tccatgacgttcctgacgtt-3′ SEQ ID No. 1.

attached to the following LNA sequence, optionally through one or more nucleotidic linker residue:

5′-GGAAGGAAGGAAGG-3′, SEQ ID No. 2.

Preferably the LNA-CpG sequence is:

5′-tccatgacgttcctgacgttXGGAAGGAAGGAAGG-3′ SEQ. ID No. 3

wherein X can be A, G, T or C, but preferably T.

In a second aspect of the present invention there is provided a method of manufacturing a LNA-CpG conjugate comprising co-synthesising the entire sequence. Preferably the oligonucleotide produced by this method is SEQ ID No.3. Conjugates formed by the method of the present invention also form an aspect of the present invention.

In eihter aspect of the present invention the preferred LNA-CpG sequences are SEQ ID NO. 3, 14, 18 or 20.

Also forming an aspect of the present invention are methods of producing DNA plasmid vaccines by binding these LNA/CpG conjugates to plasmid DNA containing a gene under the control of a promoter such that the gene may be expressed in vivo. The INA conjugate is stable and can be administered in vivo with the plasmid DNA allowing co-localisation of the plasmid and the CpG within the cells whilst still retaining the ability of the gene to be expressed. LNA oligonucleotides, advantageously are not subject to degradation by intracellular Dnase enzymes, (Dwaine A. Braasch and David R. Corey, Chemistry and Biology 8 (2001) 1-7).

The LNA conjugate produced by the method of the second aspect of the present invention comprises an oligonucleotide of between 7-25, preferably 10-20, more preferably 11-15 bases at least one of which is a locked nucleic acid preferably at least half, more preferably the entire oligonucleotide is made of LNA bases. Typically, at least a sequence of at least 13 LNA residues is preferred for optimal stability, when bound to the plasmid DNA. Preferred LNA molecules for use in the second aspect of the present invention are listed in Table 1 or Table 2. Particularly preferred LNA oligonucleotides are shown in table 1 as LNA 11701, or SEQ ID No.2. The LNA oligonucleotide should be free from self-complementary base-pairing sequences for optimal binding to DNA. In this respect, a theoretical sequence of CpG-LNA can be analysed for its ability to. self-anneal and also to form secondary structures, using the OligoDesign programme described in Tolstrup et al, Nucleic Acids Research, 31: 3758-3762 (2003) the detailed description of the method described therein is fully incorporated herein by reference. It is preferred that the self hybridisation scores (SHS) and secondary structure scores (SSS) determined using that programme are low enough (in the sense that the composition is such that its ability to self-anneal or form secondary structures is low enough) to prevent interference with plasmid binding or CpG adjuvant activity. Accordingly, it is preferred that the SHS is below 45, more preferably below 40 and most preferably below 35. It is also preferred that the SHS is below 60, more preferably below 45 and most preferably below 30.

An alternative embodiment can be envisaged where complementary sequences to further LNA oligonucleotides are present in intial bound LNA oligonucleotides such that an array of LNA oligonucleotide can be bound to a single LNA complementary site within DNA, formed by LNA: LNA hybridization between LNA oligonucleotides.

In the present invention the LNA is conjugated to a CpG immunostimulatory moiety, so that once the LNA/CpG is associated with the DNA plasmid encoding a gene of interest and administered to a host, the DNA plasmid can express the gene and allow the function of the attached moiety. LNA and CpG, and direct co-synthesis of the two may be performed by methods as described in Verma, S. & Eckstein F. Annual Review of Biochemistry 1998, 67: 99-134, Modified oligonucleotides: synthesis and strategy for users. ed. M. J. Gait. Oligonucleotide synthesis: a practical approach-book IRL/Oxford University Press 1990: Ed: Sudhir Agrawal. Methods in Molecular Biology Vol 20: Protocols for oligonucleotides and analogs, synthesis and properties- book Humana Press, 1993.

In the context of the second aspect of the present invention, particularly preferred adjuvants for linking to DNA plasmids via the LNA are CpG oligo- and di-nucleotides, (65, 66). The CpG immunostimulatory sequence is often: Purine, Purine, C, G, pyrimidine, pyrimidine; wherein the dinucleotide CG motif is not methylated. The preferred oligonucleotides for use in adjuvants or vaccines of the present invention preferably contain two or more dinucleotide CpG motifs separated by at least three, more preferably at least six or more nucleotides. The oligonucleotides of the present invention are typically deoxynucleotides. In a preferred embodiment the intemucleotide in the oligonucleotide is phosphorodithi-oate, or more preferably a phosphorothioate bond, although phosphodiester and other intemucleotide bonds are within the scope of the invention including oligonucleotides with mixed intemucleotide linkages. Methods for producing phosphorothioate oligonucleotides or phosphorodithioate are described in U.S. Pat. No. 5,666,153, U.S. Pat. No. 5,278,302 and WO95/26204. Examples of preferred oligonucleotides have the following sequences. The sequences preferably contain phosphorothioate modified internucleotide linkages.

OLIGO 1 (SEQ ID NO:4):
TCC ATG ACG TTC CTG ACG TT (CpG 1826)
OLIGO 2 (SEQ ID NO:5):
TCT CCC AGC GTG CGC CAT (CpG 1758)
OLIGO 3 (SEQ ID NO:6):
ACC GAT GAC GTC GCC GGT GAC GGC ACC ACG
OLIGO 4 (SEQ ID NO:1):
TCG TCG TTT TGT CGT TTT GTC GTT (CpG 2006)
OLIGO 5 (SEQ ID NO:7):
TCC ATG ACG TTC CTG ATG CT (CpG 1668)

Alternative CpG oligonucleotides may comprise the preferred sequences above in that they have inconsequential deletions or additions thereto. The CpG oligonucleotides utilised in the present invention may be synthesised by any method known in the art (e.g. EP 468520). Conveniently, such oligonucleotides may be synthesised utilising an automated synthesiser.

The oligonucleotides utilised in the present invention are typically deoxynucleotides. In a preferred embodiment the intemucleotide bond in the oligonucleotide is phosphorodithioate, or more preferably phosphorothioate bond, although phosphodiesters are within the scope of the present invention. Oligonucleotide comprising different internucleotide linkages are contemplated, e.g. mixed phosphorothioate phosphodiesters. Other intemucleotide bonds which stabilise the oligonucleotide may be used.

In the first aspect of the present invention the CpG may be chemically conjugated to the LNA using any of a range of commercially available cross-linking reagents. The examples described below are by no means exhaustive and include utilisation of the amino, aryl, carboxyl and hydroxyl groups found on peptides or proteins and have been extensively reviewed, (2). Other heterobifunctional cross-linking reagents are available for coupling such reactive groups including carbodiimide cross-linkers to couple carboxyl groups to amines, eg. 1-ethyl-3-(3-dimethylaminopropyl)-carbodiimide hydrochloride and other cross-liking reagents that couple to sulphydryl groups, (eg. haloacetyls or pyridyl disuphide), or amino groups, eg. imidoesters or N-hydrosuccinimide-esters including succimidyl (maleimidomethyl) cyclohexane-1-carboxylate (SMCC) and succimidyl-4-(p-maleimidophenyl)-butyrate (SMPB).

One preferred embodiment in either of the first two aspects this invention is to design the linkage of the CpG to the LNA oligonucleotide such that it can be selectively cleaved, (perhaps in order to exert a biological response), from the LNA oligonucleotide and bound plasmid once they have been delivered to a cell. One such example of this is where the CpG adjuvant, as a phosphorothioate oligonucleotide, is linked to an LNA oligonucleotide by a single DNA phosphoramidate residue, which leaves the ‘hybrid’ oligonucleotide available for cleavage by cellular phosphodiester ezymes upon delivery to the endosomal comparment of the cell. Cleavage could then release the CpG adjuvant as a free phosphorothioate oligonucleotide to exert its biological effect.

In a preferred embodiment of the present invention the LNA-conjugate is associated with a DNA molecule encoding a gene, said DNA molecule having a sequence complementary to the LNA olignucleotide. The DNA is preferably in the form of a plasmid and preferably encodes an antigen or therapeutic protein.

The plasmid is preferably without a functional origin of replication in order to prevent plasmid replication in the host to which it is administered. The promoter is preferably a strong viral promoter such as a CMV promoter.

The plasmid can be provided with a plurality of LNA complementary binding sequences to enable a plurality of LNA/conjugates to bind. The conjugates may have discrete different functional moities. Thus in one aspect of the invention the plasmid may bind to an LNA linked to a nuclear localisation peptide and an LNA linked to a small molecule adjuvant. Typically the plasmid will be provided with 4 or more complementary LNA binding sequences preferably 10 to 20 sequences, but up to 100 sequences are possible. Accordingly in one aspect of the invention there is provided a plasmid LNA conjugate complex wherein there is at least four LNA conjugates bound to the plasmid.

In a preferred embodiment the antigen is capable of eliciting an immune response against a human pathogen, which antigen or antigenic composition is derived from HIV-1, (such as tat, nef, gp120 or gp160, gp40, p24, gag, env, vif, vpr, vpu, rev), human herpes viruses, such as gH, gL gM gB gC gK gE or gD or derivatives thereof or Immediate Early protein such as ICP27, ICP 47, IC P 4, ICP36 from HSV1 or HSV2, cytomegalovirus, especially Human, (such as gB or derivatives thereof), Epstein Barr virus (such as gp350 or derivatives thereof), Varicella Zoster Virus (such as gpI, II, III and IE63), or from a hepatitis virus such as hepatitis B virus (for example Hepatitis B Surface antigen or Hepatitis core antigen or pol), hepatitis C virus antigen and hepatitis E virus antigen, or from other viral pathogens, such as paramyxoviruses: Respiratory Syncytial virus (such as F and G proteins or derivatives thereof), or antigens from parainfluenza virus, measles virus, mumps virus, human papilloma viruses (for example HPV6, 11,16, 18, eg L1, L2, El, E2, E3, E4, E5, E6, E7), flaviviruses (e.g. Yellow Fever Virus, Dengue Virus, Tick-borne encephalitis virus, Japanese Encephalitis Virus) or hnfluenza virus cells, such as HA, NP, NA, or M proteins, or combinations thereof), or antigens derived from bacterial pathogens such as Neisseria spp, including N. gonorrhea and N. meningitidis, eg, transferrin-binding proteins, lactoferrin binding proteins, PilC, adhesins); S. pyogenes (for example M proteins or fragments thereof; C5A protease, S. agalactiae, S. mutans; H. ducreyi; Moraxella spp, including M catarrhals, also known as Branhamella catarrhalis (for example high and low molecular weight adhesins and invasins); Bordetella spp, including B. pertussis (for example pertactin, pertussis toxin or derivatives thereof, filamenteous hemagglutinin, adenylate cyclase, fimbriae), B. parapertussis and B. bronchiseptica; Mycobacterium spp., including M. tuberculosis (for example ESAT6, Antigen 85A, -B or -C, MPT 44, MPT59, MPT45, HSP10,HSP65, HSP70, HSP 75, HSP90, PPD 19kDa [Rv3763], PPD 38kDa [Rv0934]), M. bovis, M leprae, M. avium, M. paratuberculosis, M. smegmatis; Legionella spp, including L. pneumophila; Escherichia spp, including enterotoxic E. coli (for example colonization factors, heat-labile toxin or derivatives thereof, heat-stable toxin or derivatives thereof), enterohemorragic E. coli, enteropathogenic E. coli (for example shiga toxin-like toxin or derivatives thereof); Vibrio spp, including V. cholera (for example cholera toxin or derivatives thereof); Shigella spp, including S. sonnei, S. dysenteriae, S. flexnerii; Yersinia spp, including Y. enterocolitica (for example a Yop protein), Y. pestis, Y. pseudotuberculosis; Campylobacter spp, including C. jejuni (for example toxins, adhesins and invasins) and C. coli; Salmonella spp, including S. typhi, S. paratyphi, S. choleraesuis, S. enteritidis; Listeria spp., including L. monocytogenes; Helicobacter spp, including H. pylori (for example urease, catalase, vacuolating toxin); Pseudomonas spp, including P. aeruginosa; Staphylococcus spp., including S. aureus, S. epidermidis; Enterococcus spp., including E. faecalis, E. faecium; Clostridium spp., including C. tetani (for example tetanus toxin and derivative thereof), C. botulinum (for example botulinum toxin and derivative thereof), C. difficile (for example clostridium toxins A or B and derivatives thereof); Bacillus spp., including B. anthracis (for example botulinum toxin and derivatives thereof); Corynebacterium spp., including C. diphtheriae (for example diphtheria toxin and derivatives thereof); Borrelia spp., including B. burgdorferi (for example OspA, OspC, DbpA, DbpB), B. garinii (for example OspA, OspC, DbpA, DbpB), B. afzelii (for example OspA, OspC, DbpA, DbpB), B. andersonii (for example OspA, OspC, DbpA, DbpB), B. hermsii; Ehrlichia spp., including E. equi and the agent of the Human Granulocytic Ehrlichiosis; Rickettsia spp, including R. rickettsii; Chlamydia spp., including C. trachomatis (for example MOMP, heparin-binding proteins), C. pneumoniae (for example MONT, heparin-binding proteins), C. psittaci; Leptospira spp., including L. interrogans; Treponiema spp., including T. pallidum (for example the rare outer membrane proteins), T. denticola, T. hyodysenteriae; or derived from parasites such as Plasmodium spp., including P. falciparum; Toxoplasma spp., including T. gondii (for example SAG2, SAG3, Tg34); Entamoeba spp., including E. histolytica; Babesia spp., including B. microti; Trypanosoma spp., including T. cruzi; Giardia spp., including G. lamblia; Leshmania spp., including L. major; Pneumocystis spp., including P. carinii; Trichomonas spp., including T. vaginalis; Schisostoma spp., including S. mansoni, or derived from yeast such as Candida spp., including C. albicans; Cryptococcus spp., including C. neoformans.

Other preferred specific antigens for M. tuberculosis are for example Rv2557, Rv2558, RPFs: Rv0837c, Rvl884c, Rv2389c, Rv2450, Rv1009, aceA (Rv0467), PstSl, (Rv0932), SodA (Rv3846), Rv2031c 16kDal., Th Ra12, Th H9, Th Ra35, Tb38-1, Erd 14, DPV, MTI, MSL, mTTC2 and hTCC1 (WO 99/51748). Proteins for M. tuberculosis also include fusion proteins and variants thereof where at least two, preferably three polypeptides of M. tuberculosis are fused into a larger protein. Preferred fusions include Ra12-TbH9-Ra35, Erd14-DPV-MTL DPV-MTI-MSL, Erd14-DPV-MTI-MSL-mTCC2, Erd14-DPV-MTI-MSL, DPV-MTI-MSLmTCC2, TbH9-DPV-MTI (WO 99/51748).

Most preferred antigens for Chlamydia include for example the High Molecular Weight Protein (HWMP) (WO 99/17741), ORF3 (EP 366 412),,and putative membrane proteins (Pmps). Other Chlamydia antigens of the vaccine formulation can be selected from the group described in WO 99/28475.

Preferred bacterial vaccines comprise antigens derived from Streptococcus spp, including S. pneumoniae (PsaA, PspA, streptolysin, choline-binding proteins) and the protein antigen Pneumolysin (13iochem Biophys Acta, 1989, 67, 1007; Rubins et al., Microbial Pathogenesis, 25, 337-342), and mutant detoxified derivatives thereof (WO 90/06951; WO 99/03884). Other preferred bacterial vaccines comprise antigens derived from Haemophilus spp., including H. influenzae type B (for example PRP and conjugates thereof), non typeable H. influenzae, for example OMP26, high molecular weight adhesins, P5, P6, protein D and lipoprotein D, and fimbrin and fimbrin derived peptides (U.S. Pat. No. 5,843,464) or multiple copy varients or fusion proteins thereof.

The antigens that may be used in the present invention may further comprise antigens derived from parasites that cause Malaria. For example, preferred antigens from Plasmodia falciparum include RTS,S and TRAP. RTS is a hybrid protein comprising substantially all the C-terminal portion of the circumsporozoite (CS) protein of P. falciparum linked via four amino acids of the preS2 portion of Hepatitis B surface antigen to the surface (S) antigen of hepatitis B virus. It's full structure is disclosed in the International Patent Application No. PCT/EP92/02591, published under Number WO 93/10152 claiming priority from UK patent application No.9124390.7. When expressed in yeast RTS is produced as a lipoprotein particle, and when it is co-expressed with the S antigen from HBV it produces a mixed particle known as RTS,S. TRAP antigens are described in the International Patent Application No. PCT/GB89/00895, piblished under WO 90/01496. A preferred embodiment of the present invention is a Malaria vaccine wherein the antigenic preparation comprises a combination of the RTS, S and TRAP antigens. Other plasmodia antigens that are likely candidates to be components of a multistage Malaria vaccine are P. faciparum MSP1, AMA1, MSP3, EBA, GLURP, RAP1, RAP2, Sequestrin, PfEMP1, Pf332, LSA1, LSA3, STARP, SALSA, PfiEXP1, Pfs25, Pfs28, PFS27/25, Pfs16, Pfs48/45, Pfs230 and their analogues in Plasmodium spp.

The invention contemplates the use of an anti-tumour antigen and be useful for the immunotherapeutic treatment of cancers. For example, tumour rejection antigens such as those for prostrate, breast, colorectal, lung, pancreatic, renal or melanoma cancers. Exemplary antigens include MAGE 1, 3 and MAGE 4 or other MAGE antigens such as disclosed in WO99/40188, PRAME, BAGE, Lage (also known as NY Eos 1) SAGE and HAGE (WO 99/53061) or GAGE (Robbins and Kawakami, 1996, Current Opinions in Immunology 8, pps 628-636; Van den Eynde et al., International Journal of Clinical & Laboratory Research (submitted 1997); Correale et al. (1997), Journal of the National Cancer Institute 89, p293. Indeed these antigens are expressed in a wide range of tumour types such as melanoma, lung carcinoma, sarcoma and bladder carcinoma.

MAGE antigens for use in the present invention may be expressed as a fusion protein with an expression enhancer or an Immunological fusion partner. In particular, the Mage protein may be fused to Protein D from Heamophilus influenzae B. In particular, the fusion partner may comprise the first ⅓ of Protein D. Such constructs are disclosed in Wo99/40188. Other examples of fusion proteins that may contain cancer specific epitopes include bcr/abl fusion proteins.

In a preferred embodiment prostate antigens are utilised, such as Prostate specific antigen (PSA), PAP, PSCA (PNAS 95(4) 1735-1740 1998), PSMA or antigen known as Prostase.

Prostase is a prostate-specific serine protease (trypsin-like), 254 amino acid-long, with a conserved serine protease catalytic triad H-D-S and a amino-terminal pre-propeptide sequence, indicating a potential secretory function (P. Nelson, Lu Gan, C. Ferguson, P. Moss, R. Gelinas, L. Hood & K. Wand, “Molecular cloning and characterisation of prostase, an androgen-regulated serine protease with prostate restricted expression, In Proc. Natl. Acad. Sci. USA (1999) 96, 3114-3119). A putative glycosylation site has been described. The predicted structure is very similar to other known serine proteases, showing that the mature polypeptide folds into a single domain. The mature protein is 224 amino acids-long, with one A2 epitope shown to be naturally processed.

Prostase nucleotide sequence and deduced polypeptide sequence and homologs are disclosed in Ferguson, et al. (Proc. Natl. Acad. Sci. USA 1999, 96, 3114-3119) and in International Patent Applications No. WO 98/12302 (and also the corresponding granted U.S. Pat. No. 5,955,306), WO 98/20117 (and also the corresponding granted U.S. Pat. No. 5,840,871 and U.S. Pat. No. 5,786,148) (prostate-specific kallikrein) and WO 00/04149 (P703P).

The present invention provides antigens comprising prostase protein fusions based on prostase protein and fragments and homologues thereof (“derivatives”). Such derivatives are suitable for use in therapeutic vaccine formulations which are suitable for the treatment of a prostate tumours. Typically the fragment will contain at least 20, preferably 50, more preferably 100 contiguous amino acids as disclosed in the above referenced patent and patent applications.

A further preferred prostate antigen is known as P501S, sequence ID no 113 of WO98/37814. Immunogenic fragments and portions encoded by the gene thereof comprising at least 20, preferably 50, more preferably 100 contiguous amino acids as disclosed in the above referenced patent application, are contemplated. A particular fragment is PS108 (WO 98/50567).

Other prostate specific antigens are known from WO98/37418, and WO/004149. Another is STEAP PNAS 96 14523 14528 7-12 1999.

Other tumour associated antigens useful in the context of the present invention include: Plu-1 J Biol. Chem 274 (22) 15633-15645, 1999, HASH-1, HasH-2, Cripto (Salomon et al Bioessays 199, 21 61-70,U.S. Pat. No. 5,654,140) Criptin U.S. Pat. No. 5,981,215, ., Additionally, antigens particularly relevant for vaccines in the therapy of cancer also comprise tyrosinase and survivin.

The present invention is also useful in combination with breast cancer antigens such as Muc-1, Muc-2, EpCAM, her 2/Neu, mammaglobin (U.S. Pat. No. 5,668,267) or those disclosed in WO/00 52165, WO99/33869, WO99/19479, WO 98/45328. Her 2 neu antigens are disclosed inter alia, in U.S. Pat. No. 5,801,005. Preferably the Her 2 neu comprises the entire extracellular domain (comprising approximately amino acid 1-645) or fragmants thereof and at least an immunogenic portion of or the entire intracellular domain approximately the C terminal 580 amino acids. In particular, the intracellular portion should comprise the phosphorylation domain or fragments thereof. Such constructs are disclosed in WO00/44899. A particularly preferred construct is known as ECD PD a second is known as ECD APD. (See WO/00/44899.) The her 2 neu as used herein can be derived from rat, mouse or human.

The plasmid may encode antigens associated with tumour-support mechanisms (e.g. angiogenesis, tumour invasion) for example tie 2, VEGF.

Vaccines of the present invention may also be used for the prophylaxis or therapy of chronic disorders in addition to allergy, cancer or infectious diseases. Such chronic disorders are diseases such as asthma, atherosclerosis, and Alzheimers and other auto-immune disorders. Vaccines for use as a contraceptive may also be considered.

Antigens relevant for the prophylaxis and the therapy of patients susceptible to or suffering from Alzheimer neurodegenerative disease are, in particular, the N terminal 39-43 amino acid fragment (A. the amyloid precursor protein and smaller fragments. This antigen is disclosed in the International Patent Application No. WO 99/27944—(Athena Neurosciences).

Potential self-antigens that could be included as vaccines for auto-immune disorders or as a contraceptive vaccine include: cytokines, hormones, growth factors or extracellular proteins, more preferably a 4-helical cytokine, most preferably IL13. Cytokines include, for example, IL1, IL2, IL3, IL4, IL5, IL6, IL7, IL8, IL9, IL10, IL11, IL12, IL13, IL14, IL15, IL16, IL17, IL18, IL20, IL21, TNF, TGF, GMCSF, MCSF and OSM. 4-helical cytokines include IL2, IL3, IL4, IL5, IL13, GMCSF and MCSF. Hormones include, for example, luteinising hormone (LH), follicle stimulating hormone (FSH), chorionic gonadotropin (CG), VGF, GHrelin, agouti, agouti related protein and neuropeptide Y. Growth factors include, for example, VEGF.

The vaccines of the present invention are particularly suited for the immunotherapeutic treatment of diseases, such as chronic conditions and cancers, but also for the therapy of persistent infections. Accordingly the vaccines of the present invention are particularly suitable for the immunotherapy of infectious diseases, such as Tuberculosis (TB), AIDS and Hepatitis B (HepB) virus infections.

Accordingly there is provided vaccines comprising the present invention for the immunotherapy of infectious diseases such as TB, AIDS and HepB; and their use in the manufacture of medicaments for the immunotherapy of infectious diseases such as TB, AIDS and HepB. In the context of TB, there is provided a method of treating an individual suffering from TB infection, comprising the administration of a vaccine of the present invention to the individual, thereby reducing the bacterial load of that individual. The reduction of bacterial load, consisting of a reduction of the amount of TB found in the lung sputum, leading to the amelioration or cure of the TB disease.

Also, in the context of AIDS, there is provided a method of treatment of an individual susceptible to or suffering from AIDS. The method comprising the administration of a vaccine of the present invention to the individual, thereby reducing the amount of CD4+ T-cell decline caused by subsequent HIV infection, or slowing or halting the CD4+ T-cell decline in an individual already infected with HIV.

Additionally, in the context of persistant Hepatitis B virus infection, there is provided a method of treatment of an individual susceptible to or suffering from HepB infection. Accordingly, there is provided a method comprising the administration of a vaccine of the present invention to the individual, thereby reducing the level of HepB load in the serum (as measured by DNA clearance) and also reducing the amount of liver damage (as detected by the reduction or stabilisation of serum levels of the enzyme Alanine Transferase (ALT)).

The LNA-CpG/DNA complex may thus be formulated into a pharmaceutical or immunogenic composition or vaccine. In an embodiment of the invention, a polynucleotide is administered/delivered as “naked” DNA, for example as described in Ulmer et al., Science 259:1745-1749, 1993 and reviewed by Cohen, Science 259:1691-1692, 1993. Here the DNA is formulated in a buffered saline solution and injected directly into tissue. The uptake of naked DNA may be increased by coating the DNA onto biodegradable beads, which are efficiently transported into the cells or by using other well known transfection facilitating agents. LNA-conjugate/DNA may be administered in conjunction with a carrier such as, for example, liposomes. Typically such liposomes are cationic, for example imidazolium derivatives (WO95/14380), guanidine derivatives (WO95/14381), phosphatidyl choline derivatives (WO95/35301), piperazine derivatives (WO95/14651) and biguanide derivatives. The LNA-CpG/DNA complex may deliver a gene of interest such as CTFR or erythropoetin gene operatively linked to a promoter sequence. Thus a method of correcting or compensating for a disease or disorder whose etiology is characterised by a genetic aberration (such as cystic fibrosis) is provided, which method comprises the step of administrating to a mammalian patient in clinical need thereof atherapeutically effective amount of the construct, preferably incorporated into a carrier.

In still another embodiment, a composition of the present invention can be delivered via a particle bombardment approach, many of which have been described (WO 91/07487). In one illustrative example, gas-driven particle acceleration can be achieved with devices such as those manufactured by Powderject Pharmaceuticals PLC (Oxford, UK) and Powderject Vaccines Inc. (Madison, Wis.), some examples of which are described in U.S. Pat. Nos. 5,846,796; 6,010,478; 5,865,796; 5,584,807; and EP Patent No. 0500 799. This approach offers a needle-free delivery approach wherein a dry powder formulation of microscopic particles, such as polynucleotide, are accelerated to high speed within a helium gas jet generated by a hand held device, propelling the particles into a target tissue of interest, typically the skin. The particles are preferably gold beads of a 0.4-4.0 um, more preferably 0.6-2.0 um diameter and the DNA conjugate coated onto these and then encased in a cartridge for placing into the “gene gun”.

Accordingly, there is provided a DNA delivery device comprising dense microbeads coated with DNA plasmid encoding a gene of interest, which plasmid is associated with one or more LNA linked to CpG compositions of the present invention. Preferably there is provided a vaccine or immunogenic composition comprising CpG-LNA-plasmid adsorbed gold microbeads.

In a related embodiment, other devices and methods that may be useful for gas-driven needle-less injection of compositions of the present invention include those provided by Bioject, Inc. (Portland, Oreg.), some examples of which are described in U.S. Pat. Nos. 4,790,824; 5,064,413; 5,312,335; 5,383,851; 5,399,163; 5,520,639 and 5,993,412.

The invention is illustrated by, but not limited to, the following examples.

### EXAMPLE 1

### Investigation of LNA Oligonucleotide Sequence Requirements and Conditions for Binding to Supercoiled Plasmid DNA Plasmids

Supercoiled plasmids that can be used in this study are plasmid pGG2XGFP a GFP expression vector, (Gene Therapy Systems [GTS], Inc., San Diego, Calif., USA), this contains multiple AG motifs within the DNA sequence of the bGHpyA region of the plasmid and multiple AAGG motifs within the DNA sequence 5′ to the CMV promoter, (GTS Catalogue 2002, 50).

Plasmid pGG2XEMPTY is an expression vector, derived from pGG2XGFP by deletion of the GFP gene, but retaining a polylinker for the insertion of a gene of interest, to be expressed under the control of the CMV promoter. To construct plasmid pGG2XEMPTY from plasmid pGG2XGFP, the latter was digested with the restriction enzymes Nhe I and BamH I, (New England Biolabs, [NEB], Hitchin, Herts., UK), to delete the region encoding the GFP gene and the remaining 5.1kb plasmid fragment was gel purified, treated with Klenow DNA Polymerase, (NEB), and ligated together using T4 DNA ligase, (NEB), prior introduction in to E. coli, (51). Bacterial cells containing plasmid pGG2XEMPTY were identified by standard procedures, (51).

LNA Oligonucleotides Used In This Study

The LNA oligonucleotides used in this study are described in Table 1

TABLE 1
lists oligonucleotide sequences used in this study.

| | | | | |
|---|---|---|---|---|
| | SEQ | No. | | |
| | ID | of | | |
| Name | NO. | sites | Description | Sequence |

| | | | | |
|---|---|---|---|---|
| 5876 | 8 | 6 | 13 mer 100% | 5′-NH₂-CTCTCTCTCTCTC-3′ |
| | | | LNA | |
| 5877 | 9 | 5 | 14 mer 100% | 5′-NH₂-CCTTCCTTCCTTCC-3′ |
| | | | LNA | |
| 5827 | 10 | 6 | 13 mer 100% | 5′-NH₂-GAGAGAGAGAGAG-3′ |
| | | | LNA | |
| 5875 | 11 | 6 | 11 mer 100% LNA | 5′-NH₂-CTCTCTCTCTC-3′ |
| 5747 | 12 | 8 | 9 mer ‘bis’ 50% | 5′-NH₂-CtCtCtCtC-XXX- |
| | | | LNA | CtCtCtCtC-3′ |
| 6563 | 13 | 6 | 11 mer 100% LNA | 5′-TAMRA-CTCTCTCTCTC-3′ |
| 11701 | 2 | 5 | 14 mer 100% LNA | 5′-NH₂-GGAAGGAAGGAAGG-3′ |
| PTOCpG | 14 | 6 | 21 mer DNA 13 mer | 5′ [symbol] tGAGAG |
| | | | LNA | AGAGAGAG-3′ |
| PTOGpC | 15 | 6 | 21 mer DNA 13 mer | 5′ [symbol] tGAGAG |
| | | | LNA | AGAGAGAG-3′ |
| 5′SHGA | 16 | 6 | 13 mer 100% LNA | 5′-S-S-GAGAGAGAGAGAG-3′ |

LNA residues are displayed in bold upper case, DNA residues are shown in bold lower case with PTO residues additionally italicised. Number of sites refers to the maximum number of theoretical oligonucleotide binding sites present on either the gWiz or pGG2XGFP, plasmid.
X=‘PEG spacer’-9-O-Dimethoxytrityl-triethylene glycol, 1-[(2-cyanoethyl)-(N,N-diisopropyl)]-phosphoramidite, spacer phosphoramidate 9, (Glen Research, USA); O=8-amino-3,6-dioxaoctanoic acid linker, g=glycine, l=lysine, Fl=Fluorescein, NH₂=5′-amino-modifier C12 phosphoramidite spacers, (Glen Research, USA), PTO=phosphorothioate, S—S=Thiol modifier, C6 S—S phosphoramidate, (Glen Research, USA).

All LNA oligonucleotides were synthesized by Proligo LLC, Colorado, USA. The majority were made with 5′- amino-modifier C12 phosphoramidite spacers, (Glen Research, USA), to allow for labelling with Alexa Fluor dyes, (Molecular Probes, Netherlands), or heterobifunctional linkers, eg. Maleimide or SPDP, (Perbio, USA). Most are 100% LNA monomers, but LNA 5747, (Table 3), is 50% LNA and 50% DNA. This ‘bis’ LNA oligonucleotide was made as an analogue of the ‘bis’ PNA clamps described in Example 1, (50, 52), and could only be efficiently synthesized as a 50:50 mix of LNA and DNA residues.

Analysis of Conditions for Binding of LNA Oligonucleotides to Supercoiled Plasmid DNA

Annealing/labelling conditions for LNA oligonucleotides were based upon those described in the literature, (55). In order to maximise the efficiency of LNA binding to supercoiled plasmid DNA labelling was performed in a buffer containing no salt at low pH<6, (10 mM phosphate buffer, 1 mM EDTA, pH 5.8) for 16 hours. at 37° C. The low pH should enable cytosine residues to Hoogsten base pair with similar efficiency to pseudocytosine residues, (52), Initially, 10 ug of plasmid DNA was labelled in a total volume of 20 ul, where LNA oligonucleotides were present at a 20× molar excess over the maximum number of potential binding sites present in the plasmid DNA, 10 sites, (see Table 1, 50).

For the conditions described above LNAs could be detected as bound to plasmid.

### EXAMPLE 2

### Binding of ‘hybrid’ Co-synthesised Phosphorothioate, (PTO) CpG/LNA Oligonucleotide, (SEQ. LD No. 3), to Supercoiled Plasmid DNA Expressing Ovalbumin Antigen and Demonstration That Binding Does Not Interfere With Antigen Expression

Plasmids and Plasmid DNA.

Plasmid DNA was prepared using the Qiagen, (Qiagen GmbH, Hilden, Germany), MaxiPrep procedure or by the Qiagen Endofree Plasmid Maxi Kit and re-suspended in TE, (10 mM Tris-HCl, 1 mM EDTA) pH8.0 at 1 μg/μl. Plasmids were >95% supercoiled by agarose gel electrophoresis, (51).

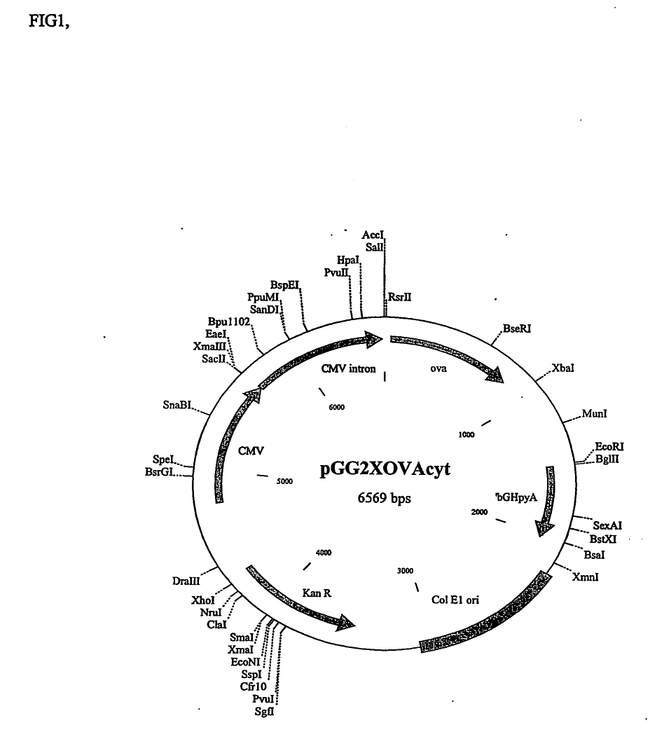

The plasmid pGG2XOVAcyt containing the cytoplasmic version of the chicken ovalbumin gene, (OVAcyt), expressed under the control of the CMV imnmediate early promoter was constructed using pVac1OVAcyt, (96), as the source of the OVAcyt gene and pGG2XEMPTY to provide the CMV promoter and binding sites for LNA oligonucleotides, (97, 98). Briefly, the polylinker of pGG2XEMPTY was cut with Sal I and the ends made flush using Klenow polymerase, the plasmid was further digested by Bgl II and treated with calf intestinal phosphatase, as described, (51). Similarly the plasmid pVac1OVAcyt was digested with NheI and Klenow treated and then digested with BglII and the 1.8kb fragment containing the OVAcyt gene was gel purified and ligated with the digested pGG2XNMPTY plasmid to produce plasmid pGG2XOVAcyt, (51). FIG. 1 shows plasmid pGG2XOVAcyt.

Oligonucleotides Used In This Study.

LNA, PTO and PTO/LNA oligonucleotides used in this study are described in Table 2; All LNA containing oligonucleotides were synthesised by Proligo LLC, Boulder, Colo., USA. All solely PTO oligonucleotides were synthesised by MWG-Biotech AG.

Endotoxin levels for any oligonucleotides transfected into RAW264.7 cells for the CpG adjuvant, TNFα induction and ELISA detection assay, (see Example 4), were obtained using either the Biowhittaker QCL-1000 LAL kit or the Pyrochrome LAL kit and were found to be less than 0.1 EU (endotoxin units)/μg of oligonucleotide.

TABLE 2
lists oligonucleotide sequences used in this study.

| | | | | |
|---|---|---|---|---|
| | SEQ | No. | | |
| | ID | of | | |
| Name | NO | sites | Description | Sequence |

| | | | | |
|---|---|---|---|---|
| CpG1826 | 1 | — | 20 mer 100% | 5′- [symbol] -3′ |
| | | | PTO | |
| GpC1745 | 17 | — | 20 mer 100% | 5′- [symbol] -3′ |
| | | | PTO | |
| PTOCpG2 | 1 | 5 | 21 mer DNA | 5′- [symbol] tGGAAGGAAG |
| | | | 14 mer LNA | GAAGG-3′ |
| PTOGpC2 | 19 | 5 | 21 mer DNA | 5′- [symbol] tGGAAGGAAG |
| | | | 14 mer LNA | GAAGG-3′ |
| PTOCpG3 | 20 | 5 | 21 mer DNA | 5′- [symbol] tGGAAGGAA |
| | | | 14 mer LNA | GGAAGG-3′ |
| PTOGpC3 | 21 | 5 | 21 mer DNA | 5′- [symbol] tGGAAGGAA |
| | | | 14 mer LNA | GGAAGG-3′ |

LNA residues are displayed in bold upper case, DNA residues are shown in bold lower case with PTO residues additionally italicised. Number of sites refers to the maximnum number of theoretical oligonucleotide binding sites present on plasmid pGG2XGFP or derivatives thereof such as pGG2XEMPTY or pGG2XOVAcyt.

Chemical Labelling of Oligonucleotides with the Ulysis Kit.

Oligonucleotides were labelled using the Ulysis nucleic acid labelling kit, (Molecular Probes, Leiden, Netherlands), containing the Alexa Fluor 488 fluorescent dye, as described, (97, 98).

Binding of Oligonucleotides to Supercoiled Plasmid DNA.

Binding of ‘hybrid’ PTO/LNA oligonucleotides, both fluorescent labelled and unlabelled, to supercoiled plasmid DNA was as described, (97, 98). Plasmid pGG2XOVAcyt was bound with PTOCpG, PTOGpC, PTOCpG2, PTOGpC2 or control binding LNA oligonucleotides: 5827, 11701 or 5′SHGA, (overnight at 37° C., 10 mM sodium phosphate, 1 mM EDTA pH 5.8). Briefly, 2.5 ug of plasmid DNA was bound with approximately 90 pmoles of Ulysis Alexa Fluor 488 or 5′ Alexa Fluor 568 labelled oligo./ug of DNA, and the resulting products were analysed on an agarose gel.

Results

Fluorescent labelled ‘hybrid’ PTO/LNA oligonucleotides containing LNA/plasmnid binding motifs based upon both the (GA)n, (PTOCPG and PTOGpC), and the (GGAA)n, (PTOCpG2 and PTOGpC2), sequences could be detected as being bound to plasmid DNA, in a similar manner to fluorescent labelled control LNAs such as 5827 or 11701 that are based upon the LNA components of the ‘hybrid’ PTO/LNA oligonucleotides. This clearly demonstrates that the 5′ extension of a 13mer 100% LNA oligonucleotide by 21 phosphorothioate and DNA residues does not interfere with the LNA's ability to bind to plasmid DNA for LNA motifs based upon both the (GA)n and (GGAA)n binding motif. The binding of the ‘hybrid’ PTO/LNA oligonucleotides to plasmid DNA under the conditions described above left some free oligonucleotide unbound to plasmid DNA as had been described previously.

Unlabelled ‘hybrid’ PTO/LNA oligonucleotides were bound to plasmid pGG2XOVAcyt, (1000 pmoles of oligonucleotide to 10 g of DNA), as described. The individual plasmid/oligonucleotide binding reactions were made up to 100 μl and free, unbound oligonucleotide was removed from plasmid and bound oligonucleotide as described below using S400HR spin columns.

Separation of Unbound PTO/LNA Oligonucleotides Using S400HR Spin Columns.

The binding of ‘hybrid’ PTO/LNA oligonucleotides to plasmid DNA under the conditions described above left some free oligonucleotide unbound to plasmid DNA. Any free oligonucleotide unbound to plasmid was removed by separation through an S400HR spin column, (Amersham Pharmacia Biotech).

Plasmid pGG2XOVAcyt with and without combinations of bound ‘hybrid’ PTO CpG, (or control GpC),/LNA oligonucleotides were transfected into CHO cells for analysis by Western Blot to determine levels of expression of OVAcyt protein expressed from pGG2XOVAcyt in the presence or absence of bound oligonucleotide. This is described in detail below.

Cell Culture and Transfection of CHO Cells

CHO K1 cells were maintained in Iscove's Modified Dulbecco's Medium, supplemented with 10% foetal calf serum, FCS), 100 units/ml penicillm, 100 ug/ml streptomycin, 2 mM glutamine, MEM nonessential amino acids and HT supplement, (Life Technologies). CHO K1 cells were grown to 80% confluence in 6 well plates, (Lab Tech, Nalge Nunc, Int.), washed twice with Optimem and transfected with plasmid DNA: cationic lipid complex, (2.5 ug plasmid DNA per well at a DNA: Transfast™,[Promega], ratio of 1 ug to 6 ul), in Optimem. Transfection mix was left in contact with the cells for 24 hours and cells were washed and topped up with growth media. Cells were harvested 48 hrs post-transfection, (after washing twice with Phosphate Buffered Saline), by re-suspension in 100 ul of boiling 2× TrisGlycine Reducing Sample Buffer, Invitrogen).

SDS/PAGE

SDS/PAGE was performed as per manufacturers instructions with 35 ul of each cell lysate, as described above, loaded on to a 4-20 % gradient Tris Glycine/SDS PAGE Novex gel, (Invitrogen, The Netherlands), with electrophoresis at a constant 125V for 90 minutes.

Western Blot Analysis

The protein was transferred from the gel to a 0.45 um PVDF transfer membrane, Immobilon-P, (Millipore), using the Novex Western Transfer appparatus following manufacturers instructions, (Invitrogen, The Netherlands). The remaining un-transferred proetin on the gel was stained up with Coomassie blue as described (51). The membrane was then blocked in 5% skimmed milk powder/0.1% Tween 20 and probed with a 1 in 500 dilution of a rabbit anti-chicken egg ovalbumin primary antibody, (Sigma). The membrane was then washed and incubated with a horse radish peroxidase conjugated goat anti-rabbit antibody, (DAKO), at a 1 in 10,000 dilution. After washing, cytoplasmic ovalbumin protein was then detected using the BioWest extended duration chemilumninescent substrate, (UVP), and visualised using the Labworks 4.0 package on the UVP EpiChemi Darkroom Bio Imaging System.

Results

The data clearly demonstrated that binding of one or two sets of ‘hybrid’ PTO/LNA oligonucleotides based on either or both of the LNA components (GA)n and (GGAA)n bound at the two regions of binding sites within plasmid pGG2XOVAcyt, (ie. within the bGHpyA and the CMV promoter), does not interfere with the expression of the antigen OVAcyt from this plasmid. This means that a CpG adjuvant can be added at two different binding sites in this system without interefering with antigen expression form the plasmid.

### EXAMPLE 3

### Optimal Design of LNA and ‘hybrid’ Co-synthesised Phosphorothioate, (PTO) CpG/LNA Oligonucleotide By Secondary Structure Prediction to Avoid Self-interaction and Maximise Efficiency of Binding to Supercoiled Plasmid DNA and Immune Adjuvant Activity

The development of a software programme, OligoDesign, (99), enables the prediction of melting temperature, self-annealing and secondary structure for LNA containing oligonucleotides. The programme includes a weighting for the increased strength of base pairing between LNA: LNA or LNA: DNA base pair interactions, compared to DNA: DNA interactions and therefore enables a more rational design of ‘hybrid’ PTO/LNA oligonucleotides, (99). Oligonucleotides that avoid predicted strong self-interactions between the PTO, (CpG), and the LNA component should perform better:—
i) as strand displacement agents, since more of the LNA will be freely accessible to bind to plasmid DNA, (97, 98),
ii) as CpG-based adjuvants, since more of the CpG sequence will be accessible, (to bind its receptor TLR9), if it is not sequestered in oligonucleotide secondary structure, (100, see Example 4).

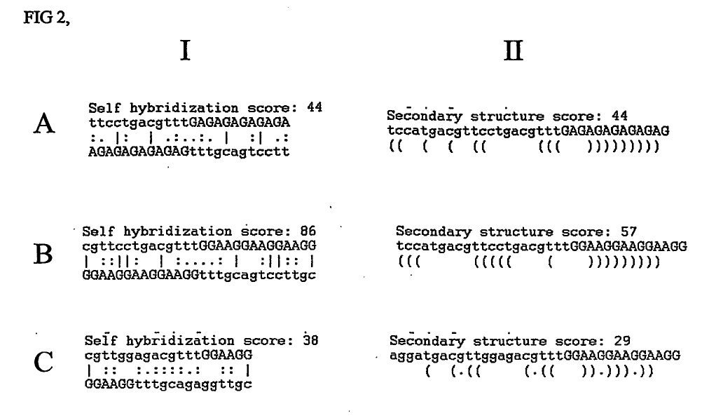

Using the OligoDesign programme, readily available in the public domain on the internet at http://lnatools.com/, ‘hybrid’ PTO/LNA oligonucleotides can have the likelihood of self-hybridization and secondary structure formation predicted. FIG. 2 shows OligoDesign predictions for the ‘hybrid’ PTO/LNA oligonucleotides described in this work.

FIG. 2 shows OligoDesign predicted intermolecular dimer, (I), formation, (self hybridization), and intramolecular secondary structure, (II), formation, (secondary structure) for ‘hybrid’ PTO/LNA oligonucleotides, with scores predicting likelihood of formation displayed,
(A)PTOCpG (SEQ ID NO. 14)
(B) PTOCpG2 (SEQ ID NO. 18)
(C) PTOCpG3 (SEQ ID NO. 20)

Note that hybridization scores below 20 are unlikely to be stable at room temperature, whereas hybridization scores above 30 are likely to be stable at room temperature. Scores below 60 can be regarded as an approximation of the melting temperature for the interaction in degrees Celsius. The lower the score the less likely the interaction is to interfere with plasmid binding, if the LNA component is involved, or CpG adjuvant activity, if the CpG motif is sequestered.

Using the known design rules for retaining CpG activity in PTO based oligonucleotides, (101, 102), the PTO sequences of ‘hybrid’ PTO/LNA oligonucleotides can be modified to reduce interaction with LNA sequences, as identified by lower self-interaction scores predicted by OligoDesign, without reducing CpG adjuvant activity. For example, for activity in mouse and murine cell lines, the retention of the 6mer ‘core’ motif: an unmethylated CpG, flanked on the 5′-side with two purine bases, and on the 3′-side with two pyrimidine bases; is required for maximum adjuvant activity, (101, 102). Other nucleotide changes outside of this ‘core’ motif have little impact on adjuvant activity. An example of such design is descnrbed below. A hybrid PTO CpG/LNA oligonucleotide, PTOCpG3, (SEQ. ID No. 4), was re-designed based upon PTOCpG2, (SEQ. ID No. 3), but reducing the predicted likelihood of intermolecular oligonucleotide dimer formation and intramolecular secondary structure formation between LNA and PTO base residues, (see FIG. 5C).

### EXAMPLE 4

### ‘Hybrid’ Co-synthesised Phosphorothioate, (PTO) CpG/LNA Oligonucleotides With Predicted Secondary Structure Suggesting Self-interaction are Poorer Immune Adjuvants Than Equivalent Doses of PTO CpG Oligonucleotide

RAW264. 7 Cell Culture, DNA Transfection and Oligonucleotide Incubation

The murine macrophage cell line RAW264.7 was maintained in RPMI 1640 medium with 10% FCS, 100 units/ml penicillin, 100 ug/ml streptomycin, 2mM glutamine, (Life Technologies). RAW264.7 cells were grown to confluence in a 96-well plate (Lab tech, Nalge Nunc. Int). Cells were washed once, with 250 μl PBS per well, and incubated in 150 ul Optimem for two hours at 37° C. A transfection mixture of 0.01-10 pM CpG oligonucleotides, ±FuGENE6 Transfection Reagent (Roche Molecular Biochemicals, at a ratio of respectively 1 uM oligonucleotide: 0.5 μl FuGENE6), in Optimem was added to a final volume of 100 μl, and the mixture was incubated at room temperature for 30 minutes. The transfection mixture was added to the RAW264.7 cells in Optimem and incubated for 14 hours at 37° C. As a control the same procedure was performed with solely FuGENE6 Transfection Reagent.

ELISA for Tumour Necrosis Factor Alpha, (TNFα), From RAW264.7 Cells.

RAW264.7 cells were grown and transfected with oligonucleotides as described above in order to perform an ELISA assay based upon production of murine TNFc after stimulation with CpG motifs, (84, 85, 86, 87).

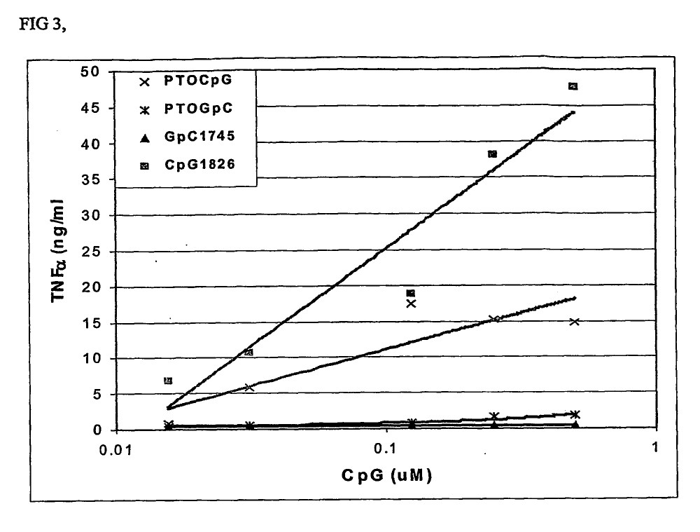

The culture supernatants were taken to detect murine TNFα levels using the Duoset ELISA development system kit, (R&D systems, Minneapolis), according to the manufacturer's protocol, after 14 hours incubation as described above. After dilution of supernatant samples in Reagent diluent, (1% BSA in PBS), the ELISA was performed in 96 well Nunc Immuno ELISA plates, (Nalge Nunc), and the absorbance was measured at 450 nm on a Molecular Devices Spectra Max 190 and the murine TNFα values were calculated using a 4-PL curve fit on the Softmax Pro 3.1.2 software. Results were expressed as mean of duplicate samples (ng/ml) and plotted against CpG oligonucleotide concentration in ELM, see FIG. 3.

PTO oligonucleotide CpG1826, (Table 2), was compared for adjuvant activity, using the in vitro RAW264.7 cell incubation assay described above, with the equivalent ‘hybrid’ PTO/LNA oligonucleotide PTOCpG, (Table 1), and the non-CpG controls: GpC1745 and PTOGpC. FIG. 3 shows aplot of ELISA data for TNFα induction in RAW264.7 cells incubated with PTO and ‘hybrid’ PTO/.LNA oligonucleotides. The data shows that although both CpG containing oligonucleotides show TNFα induction, the level of induction for equivalent concentrations of the ‘hybrid’ PTO/LNA oligonucleotide PTOCpG is much reduced compared to its PTO analogue CpG1826. The most likely explanation for this reduction in CpG activity for PTOCpG is the self-interaction predicted for this oligonucleotide that can block the accessibility of the CpG motif.

### EXAMPLE 5

Binding of ‘hybrid’ Co-synthesised Phosphorothioate, (PTO) CpG/LNA Oligonucleotide, (SEQ. ID No. 4), to Supercoiled Plasmid DNA.

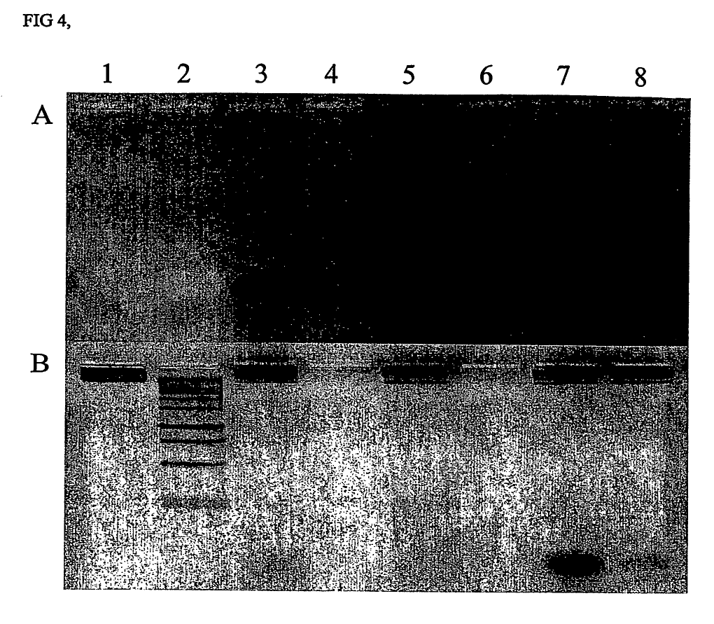

A hybrid PTO CpG/LNA oligonucleotide, PTOCpG3, (SEQ. ID No. 4), was re-designed based upon PTOCpG2, (SEQ. ID No. 3), but reducing the predicted likelihood of intermolecular oligonucleotide dimer formation and intramolecular secondary structure formation between LNA and PTO base residues, (see Example 3). Binding of fluorescent labelled ‘hybrid’ PTO/LNA oligonucleotides to supercoiled plasmid DNA is described, Examples 1 & 2, (97, 98). Plasmid pGG2XGFP was bound with PTOCpG2, PTOCpG3, (SEQ. ID No. 4), PTOGpC3 or the control binding LNA oligonucleotide: 11701, (overnight at 37° C., 10 mM sodium phosphate, 1 mM EDTA pH 5.8). Briefly, 2.5 μg of plasmid DNA was bound with approximately 90 pmoles of Ulysis Alexa Fluor 488 or 5′ Alexa Fluor 568 labelled oligo./μg of DNA, and the resulting products were analysed on an agarose gel, see FIG. 4.

FIG. 4 shows 2% agarose gel electrophoresis, in the absence of ethidium bromide, (EtBr), for analysis of supercoiled plasmid DNA incubated with LNA oligonucleotides labelled with either Alexa Fluor 568 at a 5′ NH₂ group or chemically labelled at the N⁷ G residue with a Ulysis Alexa Fluor 488 labelling kit, (Molecular Probes, Leiden, Netherlands).
(A) Gel analysis using the Labworks 4.0 package on the UVP EpiChemi Darkroom Bio Imaging System, 302 nm uv, SYBR gold filter (UVP, Cambridge, UK), before EtBr staining.
(B) As (A) after EtBr staining.
1) 2.5 ug pGG2XGFP plasmid
2) 1 ug of 1 kb DNA ladder, Promega, Southampton, UK)
3) 2.5 ug pGG2XGFP plasmid incubated with Alexa Fluor 568 labelled 11701 at 37° C.
4) Empty
5) 2.5 ug pGG2XGFP plasmid incubated with Ulysis Alexa Fluor 488 labelled PTOCpG2 at 37° C.
6) Empty
7) 2.5 ug pGG2XGFP plasmid incubated with Ulysis Alexa Fluor 488 labelled PTOCpG3 at 37° C.
8) 2.5 ug pGG2XGFP plasmid incubated with Ulysis Alexa Fluor 488 labelled PTOGpC3 at 37° C.

Both PTOCpG3, (SEQ. ID No. 4), and its non-CpG analogue PTOGpC3 bind to plasmids containing the binding site for the LNA binding motif (GGAA)n.

### OTHER PUBLICATIONS.

1) Rajur et al., 8(6):935-940 (1997).
2) Brinkley, M., 3(1):2-13 (1992).
3) Mattaj, I & Engelmeier, L., Annual Review Biochemistry 67:265-306 (1998)
4) Gorlich, D. & Kutay, U., Annual Review Cellular & Developmental Biology 15:607-660 (1999).
5) Zanta et al., Proc. Natl. Acad. Sci. USA 96: 91-96 (1999).
6) Sebestyen et al., Nature Biotech. 16: 80-85 (1998).
7) Branden et al., Nature 17: 784-787 (1999).
8) Zhang et al., Gene Therapy 6: 171-181 (1999).
9) Subramanian et al., Nature 17: 873-877 (1999).
10) Chang et al., Virology 189: 821-827 (1992).
11) Vives et al., Journal Biological Chemistry 272 (25) : 16010-16017 (1997).
12) Derossi et al., Journal Biological Chemistry 269 (14) :10444-10450 (1994).
13) Derossi et al., Trends in Cell Biology 8: 84-87 (1998).
14) Prochanitz, A., Current Opinion in Neurobiology 6: 629-634 (1996).
15) Prochiantz, A. & Theodore, L., BioEssays 17(1) : 39-44 (1995).
16) Eguchi et al., J. Biol Chem 272: 17640-17647 (1997).
17) Burglin, T. & De Robertis, E., EMBO J. 6: 2617-2625 (1987).
18) Ballas, N. & Citovsky, V., Proc. Natl. Acad. Sci. USA 94: 10723-10728 (1997).
19) Nigg, E., Nature 386: 779-787 (1997).
20) Blanke et al., Proc. Natl. Acad. Sci. USA 93: 8437-8442 (1996).
21) Morris et al., Nucleic Acids Research 27 3510-3517 (1999).
22) Schmolke et al., Journal of Virology 69: 1071-1078 (1995).
23) Wychowski et al., EMBO J. 5: 2569-2576.
24) Xia et al., Journal of Virology 66: 914-921 (1992).
25) Dean, D., Adv. Drug Delivery reviews 44: 81-95 (2000).
26) Lindgren et al., Trends in Pharmaceutical Sciences 21: 99-103 (2000).
27) Elliot, G. & O'Hare, P, Cell 88: 223-233 (1997).
28) Kawana et al., Journal of Virology 75 (5) : 2331-2336 (2001).
29) Pastan, I & Fitzgerald, D., Science 254: 1173-1177 (1991).
30) Oess, S. & Hildt, E.., Gene Therapy 7: 750-758 (2000).
31) Mechtler, K. & Wagner, E, New Journal of Chemistry 21: 105-111 (1997).
32) Pillot et al., Journal Biological Chemistry 271: 28757-28765 (1996).
33) Subbarao et al., Biochemistry 26 (11) : 2964-2972 (1987).
34) Niidome et al., Journal Biological Chemistry, 272: 15307-15312 (1997).
35) Wyman et al., Biochemistry 36: 3008-3017 (1997).
36) Vogel et al., Journal American Chemical Society 118: 1581-1586 (1996).
37) Kichler et al., Bioconjugate Chemistry 8: 213-221 (1997).
38) Puls, R. & Minchin, M., Gene Therapy 6: 1774-1778 (1999).
39) Goldman et al., Cancer Research 57: 1447-1451 (1997)
40) Kircheis et al., Gene Therapy 4: 409-418 (1997)
41) Kawakami et al., Gene Therapy 7: 292-299 (2000).
42) Fajac et al., Human Gene Therapy 10: 395-406 (1999).
43) Zanta et al., Bioconjugate Chemistry 8: 839-844 (1997).
44) Hung et al., Cancer Research 61: 1080-1088 (2001).
45) Hart et al., Gene Therapy, 2: 552-554 (1995).
46) Deonarain, M., Expert Opinion on Therapeutic Patents 8(1) : 53-69 (1998).
47) Cristiano, R. & Roth, J., Journal Molecular Medicine 73: 479-486 (1995)
48) Uherek, C. et al., Journal of Biological Chemistry, 273: 8835-8841 (1998).
49) Chattergoon et al., Nature Biotechnology 18: 974-979 (2000).
50) Zelphati et al., Human Gene Therapy 10: 15-24 (1999).
51) Maniatis et al., ‘Molecular Cloning: a laboratory manual’ 2^{nd} Edition, (1992)
52) Egholm et al., Nucleic Acids Research 23: 217-222 (1995).
53) Zhang et al., Nucleic Acids Research 28 (17) : 3332-3338 (2000).
54) Griffith et al., Journal American Chemical Society 117: 831-832, (1995).
55) Nielsen, P. & Egholm, M. ‘An introduction to PNA’ Chapter 1 In Nielsen, P. & Egholm, M (Eds.) Peptide Nucleic Acids: Protocols and Applications (1999).
56) Butz, E. & Bevan, M., Journal of Immunology 160: 2139-2144, (1998).
57) Tuting, et al., Journal of Immunology 160: 1139-1147, (1998).
58) Harrison, et al., Vaccine 19: 1820-1826, (2001).
59) Vasilakos, et al., Cellular immunology 204: 64-74 (2000).
60) Rhodes, J. et al., Nature 377: 71-75 (1995).
61) Gherardi, M. et al., Journal of Virology 74 (14) : 6278-6286.
62) Alderton, W. et al., Biochemical Journal 357: 593-615, (2001).
63) Scheerlinck, J P. ‘Genetic adjuvants for DNA vaccines’, Vaccine 19: 2647-2656, (2001).
64) Reyes et al., Vaccine 19: 3778-3786, (2001).
65) Sato, Y. et al., Science 273 (5273) : 352-354 (1996).
66) Hemmi, H. et al., Nature 408: 740-745, (2000)
67) Beutler, B., Current Opinion in Microbiology 3: 23-30 (2000).
68) Thoma-Uszynski, S. et al., Journal of Immunology 165: 3804-3810 (2000).
69) Mitchell, P. & Tijan, R, Science 245: 371-378, (1989).
70) Courey, A. & Tijan, R., Cell 55: 887-898, (1988).
71) Tanaka et al., Molecular and Cellular Biology 14:: 6046-6055, (1994).
72) Ma, J. & Ptashne, M., Cell, 51: 113-119, (1987).
73) Seipel et al., EMBO Journal 11: 4961-4968, (1992).
74) Obika et al., Tetrahedron Letters 41: 221-224, (2000).
75) Torigue et al., Journal of Biological Chemistry 276(4) : 2354-2360, (2001).
76) Smulevitch et al., Nature Biotechnology 14: 1700-1704, (1996).
77) Kichler et al., Bioconjugate Chemistry 8: 213-221, (1997).
78) Bukanov et al., Proc. Natl. Acad. Sci. USA 95: 5516-5518, (1998).
79) Klinman et al., Vaccine 17: 19-25, (1999).
80) Weeratna et al., Vaccine 18: 1755-1762, (2000).
81) Shirota et al., Journal of Immunology 167: 66-74, (2001).
82) Hacker et al., EMBO Journal 17: 6230-6240, (1998).
83) Bauer et al., Proc. Natl. Acad Sci. USA 98 (16) : 9237-9242, (2001) ; International patent application: ‘Process for high throughput screening of CpG-based immuno-agonist/antagonists’, WO02/228009A2, (2002); S. Bauer, personal communication, (unpublished observation).
84) Jin et al., Journal of Immunology 165: 5153-5160, (2000).
85) Dalpke et al., Journal of Immunology 166: 7082-7089, (2001).
86) Crabtree et al., Infection and Immunity 69(4) : 2123-2129, (2001).
87) Gao et al., Journal of Immunology 166: 6855-6860, (2001).
88) Tung, C. H. and Stein, S., Bioconjugate Chemistry 11(5): 605-618, (2000).
89) Wang et al., Bioconjugate Chemistry 8: 878-884, (1997).
90) Harrison, J. and Balasubramanian, S., Nucleic Acids Research 26 (13) : 3136-3145,(1998).
91) Stetsenko, D. and Gait, M., J. Org. Chem. 65: 49004908, (2000).
92) Stetsenko, D. and Gait, M., Nucleosides, Nucleotides &Nucleic Acids 19(10-12) : 1751-1764, (2000).
93) Stetsenko, D. and Gait, M., Bioconjugate Chemistry 12: 576-586, (2001).
94) Rojas et al., Nature Biotechnology 16: 370-375, (1998).
95) Ogris et al., Jourmal of Biochemistry 276 (50) : 47550-47555, (2001).
(96) Bjorkdahl, O. et al., Immunology 110: 170-179, (2003).
(97) Hertoghs, K. et al., Nucleic Acids Research 31: 5817-5830, (2003).
(98) Catchpole, I. WO 02/102825 A2 (2002).
(99) Tolstrup, N. et al., Nucleic Acids Research 31: 3758-3762, (2003).
(100) Kandimalla, E. et al., Biochem. & Biophysics. Res. Comm. 306: 948-953, (2003).
(101) Agrawal, S. & Kandimalla, E., Trends in Mol. Medicine 8: 114121, (2002).
(102) Kandimalla, E. et al., Curr. Opinion in Mol. Therapeutics 4:122-129, (2002).

1. An oligonucleotide comprising an unmethylated CG dinucleotide (CpG) of the following sequence: 5′-tccatgacgttcctgacgtt-3′ SEQ ID No. 1, attached to the following LNA sequence, optionally through one or more nucleotide linker residues: 5′-GGAAGGAAGGAAGG-3′, SEQ ID No. 2.

2. An oligonucleotide as claimed in claim 1 wherein the sequence is selected from SEQ ID NO. 3, SEQ IID NO. 14, SEQ ID NO. 18 or SEQ ID NO. 20.

3. A complex comprising a plasmid and attached thereto a plurality of oligonucleotides as claimed in claim 1.

4. A method of manufacturing a LNA-CpG conjugate comprising co-synthesising the entire sequence.

5. A method of manufacturing a LNA-CpG conjugate as claimed in claim 4, wherein the sequence of the LNA-CpG conjugate has sufficiently low predicted self annealing and secondary structure forming capacity to prevent inhibition of binding to a plasmid or CpG adjuvant activity.
